(19) United States
(12) Patent Application Publication
(10) Pub. No.: US 2024/0399071 A1
(43) Pub. Date: Dec. 5, 2024

(54) NEEDLE SHIELD REMOVER

(71) Applicant: Owen Mumford Limited, Woodstock, Oxfordshire (GB)

(72) Inventors: Robert William BRUNS, Woodstock, Oxfordshire (GB); David Robert BLOUNT, Woodstock, Oxfordshire (GB)

(21) Appl. No.: 18/702,224

(22) PCT Filed: Oct. 12, 2022
(86) PCT No.: PCT/EP2022/078439

§ 371 Date: Apr. 17, 2024

(30) Foreign Application Priority Data
Oct. 20, 2021 (GB) 2115006.5

(51) Int. Cl. A61M 5/32

(52) U.S. Cl. CPC A61M 5/3204

(57) ABSTRACT

Aspects of the present invention relate to a needle shield remover comprising at least one flexible elongate member, the elongate member being provided with a gripping member configured to engage a needle shield.

### TECHNICAL FIELD

The present disclosure relates to a needle shield remover. In particular, it relates to a needle shield remover, to a cap including a needle shield remover and to an injection device configured to receive a cap including a needle shield remover.

### BACKGROUND

Injection devices including a syringe, such as auto-injectors, are commonly provided with at least one needle shield to protect the syringe needle from damage and accidental contact and to maintain sterility of the injection needle. The needle shield may be a soft needle shield that is formed of a flexible material or a rigid needle shield that is formed of a rigid, inflexible material which provide greater mechanical protection to the injection needle. Conventional injection devices may also include a removable cap covering the needle shields to provide mechanical protection for the needle shields and to facilitate removal of the needle shields before an injection may be performed.

### SUMMARY OF THE INVENTION

Aspects and embodiments of the invention provide a needle shield remover, a cap including a needle shield remover and an injection device as claimed in the appended claims.

According to an aspect of the present invention there is provided a needle shield remover comprising at least one flexible elongate member; the elongate member being provided with a gripping member configured to engage a needle shield.

The needle shield remover comprises a pair of flexible elongate members which optionally form an opposing pair. Each elongate member may be provided with a gripping member configured to engage a needle shield.

The pair of elongate members may be connected by a perpendicular joining portion which optionally forms a U shape remover.

The or each elongate member may be provided with a second gripping member in a distal part of the member configured to engage with a cap.

The needle shield remover may be formed from a metal with the gripping member being formed of a portion of the metal that is bent inwardly.

A cap for an injection device including the needle shield remover described above may also be provided.

The cap may include an end wall and an outer wall that extends proximally from the end wall; the end wall including an aperture to allow the needle shield remover to be inserted into the cap.

The cap may include an end wall and an outer wall that extends proximally from the end wall; the end wall including an aperture to allow the needle shield remover to be inserted into the cap and a bridging portion which the joining portion of the needle shield remover engages with to prevent rearward movement of the needle shield remover.

Optionally, the cap may include a portion to insert into the aperture to prevent distal movement of the needle shield remover.

Optionally, where the needle shield remover is provided with a second gripping member the cap may be provided with a corresponding aperture to receive the second gripping member.

The cap may include an inner wall that extends proximally from the end wall; the flexible member being disposed along the outer surface of the inner wall.

There may also be provided an injection device comprising a cap described previously wherein the device includes a medicament container including a needle; and a needle shield wherein the injection device is configured such that when the medicament container is in the device the gripping member is positioned rearward of the needle shield.

### BRIEF DESCRIPTION OF THE DRAWINGS

One or more embodiments of the invention will now be described, by way of example only, with reference to the accompanying drawings, in which:

FIG. 1 shows

### DETAILED DESCRIPTION

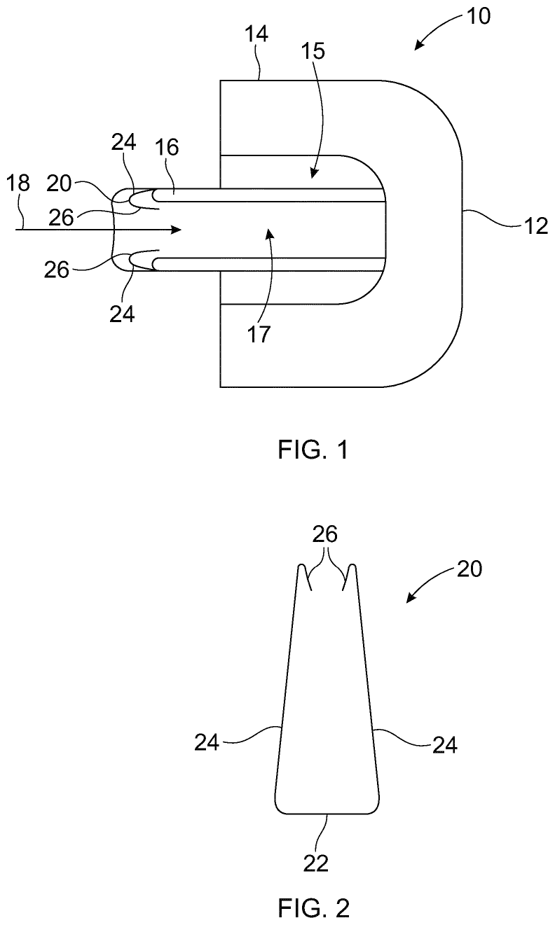

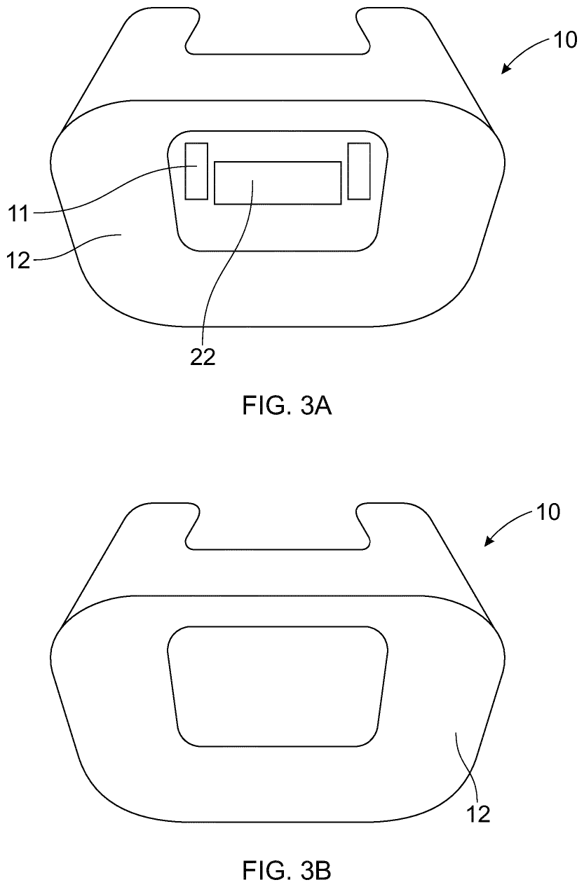

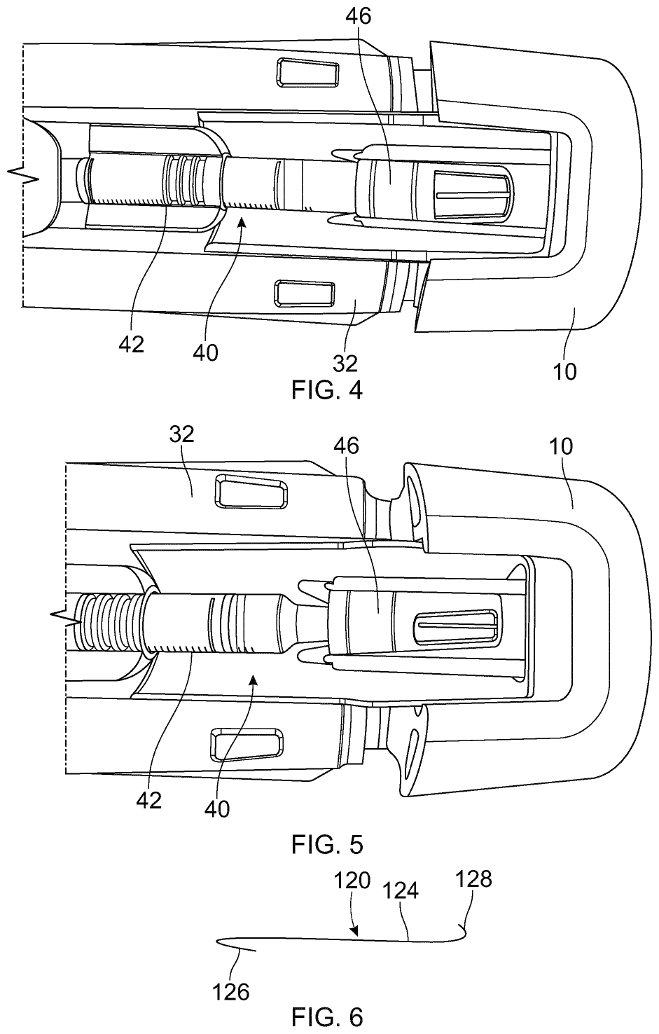

A top view of a cap 10 incorporating a needle shield remover 20 according to an embodiment of the present invention is described herein with reference to FIG. 1.

With reference to FIG. 1, the cap 10 comprises a distal end wall 12 and an outer wall 14 extending proximally from the end wall 12. The cap also has an inner wall 16 extending proximally from the end wall 12 defining a passage 18 into which a distal end of a syringe (not shown) may be inserted.

A part of the outer 14 and inner wall 16 is provided with a cut out 15 and 17 respectively. The cut out 15, 17 allows a syringe (not shown) including a medicament container, a needle and a needle shield to be inserted into the device through the cut out 15, 17 or through the open end of the cap 10. The skilled person would understand that the cut outs 15, 17 are optional and the outer and inner walls 14, 16 may be continuous such that the syringe is inserted into the cap through the open end of the cap 10.

The cap 10 further comprises a needle shield remover 20. The needle shield remover 20 is shown in more detail in FIG. 2. As can be seen the needle shield remover 20 comprises a U shaped piece of metal. The distal end 22 of the needle shield remover 20 is flat and two flexible arms 24 extend proximally from the distal part of the needle shield remover 20. The arms 24 are angled inwardly from the distal end of the needle shield remover 20 to the proximal end of the needle shield remover 20. The proximal end of each of the arms is provided with a gripping member 26 which extend inwardly and rearwardly.

FIG. 1 illustrates the needle shield remover 20 installed within the cap 10. The arms 24 of the needle shield extend along the outer surface of the inner wall 16 of the cap 10.

FIGS. 3a and 3b show the cap 10 looking directly onto the distal end wall 12. As can be seen the distal end wall is provided with apertures 11 through which the arms 24 of the removeable needle shield 20 can pass to extend along the outer surface of the inner wall 16 of the cap 10. The distal end wall is provided with a recessed area which receives the distal end 22 of the needle shield remover 20 so that it sits proximally from the distal surface of the distal end wall 12. The recessed area can receive a distal end cap 17 which is received in the distal end wall as show in in FIG. 3b in order that the distal surface of the cap's distal end 12 appears as a smooth surface. In this way the cap 10 including the needle shield remover 20 can be assembled by inserting the needle shield remover 20 into the cap through the distal end and then the distal end cap 17 is inserted into the recessed area to create the final form of the cap 10 for use on a device.

Referring now to FIG. 4 which illustrates the cap described above inserted onto a device 30 which comprises a housing 32 that is arranged to receive a syringe 40 comprising a medicament container 42, a needle (not shown) and a needle shield 46 which is placed over the needle.

As can be seen in FIG. 4 the needle shield 46 and part of the medicament container is received within the arms 24 of the needle shield remover and inner wall. The arms 24 of the needle shield remover 20 are able to flex outwards in the event that the diameter of the medicament container or part of the needle shield 46 is greater than the distance between the proximal ends of the arms 24. The passageway defined by the inner wall helps to ensure that the needle shield can pass through the gap between the arms 24 of the needle shield remover 24 and does not interact with them in any other way, for example, by bending the arms to obstruct entrance of the needle shield into the needle shield remover.

When the user comes to use the device 30 they move the cap 10 in a distal direction to remove it from the housing of the device 30. As the cap moves in the distal direction so does the needle shield remover 20. As the needle shield remover passes over the gap between the medicament container 42 and the needle shield 46 the arms are no longer held apart by the medicament container and the inward projections at the proximal end of the arms move inwards into the gap between the medicament container 42 and the needle shield to engage the needle shield 46 as shown in FIG. 5. Once the inward projections have engaged the needle shield 46 further distal movement of the cap 10 and needle shield remover 20 also causes distal movement of the needle shield 46 to remove the needle shield 46 from the syringe within the cap 10. Once the needle shield 46 has been removed from the device 30 it can be removed from the cap 10 either by pulling out the needle shield through the cut out, flexing the arms outwards or by any other suitable means. This allows the cap to be reused on the device when another syringe is inserted into the device.

An alternative embodiment of the needle shield is illustrated in FIG. 6. The needle shield 120 of the alternative embodiment includes a flexible arm 124 provided with a first gripping member 126 which extends inwardly and rearwardly and is configured to grip the removeable needle shield as described with reference to embodiment 1 above. The flexible arm 124 is further provided with another gripping member 128 at the distal end of the flexible arm. The another gripping member 128 is configured to engage with the cap of the device to hold the needle shield remover 120 in engagement with the cap. This may be, for example, by compressing into the material of the cap or, alternatively by being received in a corresponding aperture in the cap. In the alternative embodiment there may be provided any number of flexible arms, for example there may only be one flexible arm or an opposing pair of flexible arms.

Although the gripping members 26, 126 have been described as being disposed at the proximal end of each flexible arm 24, 124 the skilled person would understand that the gripping members 26, 126 may be spaced apart from the proximal end of the flexible arm 24, 124.

The skilled person would understand that although two methods of securing the needle shield remover 20, 120 to the cap have been disclosed any suitable method of securing the arms to the cap may be used. For example the cap material may be moulded over the needle shield remover.

The needle shield remover may be made of any suitable material. For example, the needle shield remover may be made from a metal with the gripping members formed by bending over parts of the needle shield remover.

Certain terminology is used in the following description for convenience only and is not limiting. The word “distal” refers to the front, or patient, end of the device. The word “proximal” refers to the rear, or syringe user, end of the device. The term “longitudinal”, with or without axis, refers to a direction on an axis through the device in the direction of the longest extension of the device. The term “radial” or “transverse/transversal”, with or without axis, refers to a direction generally perpendicular to the longitudinal direction, e.g. “radially outward” would refer to a direction pointing away from the longitudinal axis.

It should be understood that the detailed description and specific examples are given by way of illustration only, since various changes and modifications within the spirit and scope of the disclosure will become apparent to those skilled in the art from the description.

1. A needle shield remover comprising at least one flexible elongate member; the elongate member being provided with a gripping member configured to engage a needle shield.

2. A needle shield remover according to claim 1 wherein the needle shield remover comprises a pair of flexible elongate members; each elongate member being provided with a gripping member configured to engage a needle shield.

3. A needle shield remover as claimed in claim 2 wherein the pair of elongate members form an opposing pair.

4. A needle shield remover as claimed in claim 2 or claim 3 wherein the pair of elongate members are connected by a perpendicular joining portion.

5. A needle shield remover as claimed in claim 4 wherein the needle shield remover forms a U shape.

6. A needle shield remover as claimed in any one of claims 1 to 3 wherein the or each elongate member is provided with a second gripping member in a distal part of the member configured to engage with a cap.

7. A needle shield remover as claimed in any preceding claim wherein the needle shield remover is formed from a metal.

8. A needle shield remover as claimed in claim 7 wherein the gripping member comprises a portion that is bent inwardly.

9. A cap for an injection device comprising a needle shield remover as claimed in any preceding claim.

10. A cap for an injection device comprising a needle shield remover as claimed in any one of claims 1 to 5 wherein the cap comprises an end wall and an outer wall that extends proximally from the end wall; the end wall including an aperture to allow the needle shield remover to be inserted into the cap.

11. A cap for an injection device comprising a needle shield remover as claimed in any one of claims 4 or 5 wherein the cap comprises an end wall and an outer wall that extends proximally from the end wall; the end wall including an aperture to allow the needle shield remover to be inserted into the cap and a bridging portion which the joining portion of the needle shield remover engages with to prevent rearward movement of the needle shield remover.

12. A cap for an injection device as claimed in any one of claims 9 to 12 wherein the cap further comprises a portion to insert into the aperture to prevent distal movement of the needle shield remover.

13. A cap for an injection device comprising a needle shield remover as claimed in any one of claims 6 to 9 wherein the cap is provided with a corresponding aperture to receive the second gripping member.

14. A cap for an injection device as claimed in any one of claims 9 to 13 wherein the cap further comprises an inner wall that extends proximally from the end wall; the flexible member being disposed along the outer surface of the inner wall.

15. An injection device comprising a cap as claimed in any one of claims 9 to 14 wherein the device further comprises a medicament container including a needle; and a needle shield wherein the injection device is configured such that when the medicament container is in the device the gripping member is positioned rearward of the needle shield.
